(19) United States
(12) Patent Application Publication
(10) Pub. No.: US 2024/0242367 A1
(43) Pub. Date: Jul. 18, 2024

(54) IMAGE PROCESSING DEVICE, OPERATION METHOD OF IMAGE PROCESSING DEVICE, AND PROGRAM

(71) Applicant: FUJIFILM Corporation, Tokyo (JP)

(72) Inventor: Akimichi ICHINOSE, Tokyo (JP)

(73) Assignee: FUJIFILM Corporation, Tokyo (JP)

(21) Appl. No.: 18/403,670

(22) Filed: Jan. 3, 2024

(30) Foreign Application Priority Data
Jan. 17, 2023 (JP) 2023-005047

(51) Int. Cl. G06T 7/60; A61B 6/00; G06T 7/00; G16H 50/50

(52) U.S. Cl. CPC G06T 7/60; A61B 6/5217; G06T 7/0014; G16H 50/50; G06T 2207/10072; G06T 2207/30061; G06T 2207/30096

(57) ABSTRACT

Provided are an image processing device, an operation method of the image processing device, and a program in which preferable comparison is realized for a plurality of images having different acquisition times.

An image processing device receives a plurality of first images that are generated by imaging a subject and to which each of a plurality of first image conditions different from each other is applied, designates a first region of interest in the plurality of first images, acquires, for a plurality of second images that are generated by imaging the subject before capturing the first image, measurement information on the second image having a second region of interest corresponding to the first region of interest, specifies the first image included in a range of the measurement information from the plurality of first images, and measures the first region of interest in the specified first image.

### CROSS-REFERENCE TO RELATED APPLICATION

The present application claims priority under 35 U.S.C §119(a) to Japanese Patent Application No. 2023-005047 filed on Jan. 17, 2023, which is hereby expressly incorporated by reference, in its entirety, into the present application.

### BACKGROUND OF THE INVENTION

### 1. Field of the Invention

The present invention relates to an image processing device, an operation method of the image processing device, and a program.

### 2. Description of the Related Art

In general, in a case of performing follow-up observation for the progress and a healing state of a lesion part in a body of a certain patient, an image of the lesion part is acquired, and after a certain period of time has elapsed, the image of the lesion part is acquired again and the comparative interpretation is performed for two images which are acquired at different times.

JP2002-63563A discloses an image processing method of obtaining a corresponding region of interest from two or more images to be compared for the same subject and of performing the same image processing on both regions of interest.

The image processing method described in JP2002-63563A enables accurate comparison and interpretation of regions of interest of a plurality of images to be compared and improves interpretation performance of regions of interest having a correspondence relationship.

### SUMMARY OF THE INVENTION

For example, it is most important to compare the sizes of tumors in a case where the comparative interpretation of tumors is performed. In order to accurately compare the sizes of two tumors to be compared, the measurement of the tumors in the current examination needs to be performed by applying the same conditions as the image used in the past examination or conditions similar to the image used in the past examination. However, it takes time and effort to determine the condition of the image used in the current examination according to the condition of the image used in the past examination.

In the method described in JP2002-63563A, in a case where the condition of the image used in the past examination and the condition of the image used in the current examination are not the same, it is difficult to perform comparative interpretation of the image used in the past examination and the image used in the current examination, and the above problems have not been solved.

The present invention has been made in view of these circumstances, and an object of the present invention is to provide an image processing device, an operation method of the image processing device, and a program in which preferable comparison is realized for a plurality of images having different acquisition times.

An image processing device according to a first aspect of the present disclosure is an image processing device comprising one or more processors, and one or more memories that store a command to be executed by the one or more processors, in which the one or more processors are configured to: receive a plurality of first images that are generated by imaging a subject and to which each of a plurality of first image conditions different from each other is applied; designate a first region of interest in the plurality of first images; acquire, for a plurality of second images that are generated by imaging the subject before capturing the first image, measurement information on the second image having a second region of interest corresponding to the first region of interest; specify the first image included in a range of the measurement information from the plurality of first images; and measure the first region of interest in the specified first image.

According to the image processing device according to the first aspect of the present disclosure, based on the measurement information on the second image having the second region of interest corresponding to the first region of interest designated in the first image, the first image included in the range of measurement information is specified, and the first region of interest in the specified first image is measured. Accordingly, the accurate comparison between the measurement result of the first region of interest in the first image and the measurement result of the second region of interest in the second image is realized.

According to an image processing device according to a second aspect, in the image processing device according to the first aspect, the one or more processors may be configured to specify the first image having the first image condition in which a similarity indicator representing a similarity to a second image condition of the second image is a maximum, as the first image included in the range of the measurement information.

According to this aspect, even in a case where the first image having the same first image condition as the second image condition is not present, the first image for which the accurate comparison of the measurement result with the measurement result of the second image is realized can be specified.

According to an image processing device according to a third aspect, in the image processing device according to the second aspect, the one or more processors may be configured to derive the similarity indicator using at least any one of a reconstruction condition, a slice thickness representing a thickness of a tomographic layer in a tomographic image, a spatial resolution, or an imaging protocol.

According to this aspect, the first image having the first image condition similar to the second image condition can be specified based on at least any of the reconstruction condition, the slice thickness, the spatial resolution, or the imaging protocol.

According to an image processing device according to a fourth aspect, in the image processing device according to any one of the first aspect to the third aspect, the one or more processors may be configured to apply at least any one of the second image in which measurement of the second region of interest is performed or a measurement result of the second region of interest, as the measurement information.

According to this aspect, the first image for which the accurate comparison with the measurement result of the second region of interest is realized can be specified based on at least any of the second image or the measurement result of the second region of interest.

According to an image processing device according to a fifth aspect, in the image processing device according to any one of the first aspect to the fourth aspect, the one or more processors may be configured to estimate an error in a measurement result of the first region of interest with respect to a measurement result of the second region of interest, based on the first image condition and a second image condition.

According to this aspect, in the measurement of the first region of interest in the first image having the first image condition similar to the second image condition, an error of the measurement result caused by a difference between the first image condition and the second image condition can be grasped.

According to an image processing device according to a sixth aspect, in the image processing device according to the fifth aspect, the one or more processors may be configured to output an alert in a case where the estimated error exceeds a prescribed range.

According to this aspect, it is possible to consider the execution of re-measurement of the first region of interest in the first image and the execution of re-specification of the first image.

According to an image processing device according to a seventh aspect, in the image processing device according to the fifth aspect or the sixth aspect, the one or more processors may be configured to display a range of the error prescribed according to a tendency of the estimated error on a display device.

According to this aspect, the user can grasp the range of the error of the measurement of the first region of interest in the first image.

According to an image processing device according to an eighth aspect, in the image processing device according to any one of the first aspect to the seventh aspect, the one or more processors may be configured to set a first display condition applied to measurement of the first region of interest in the first image according to a second display condition applied to measurement of the second region of interest in the second image.

According to this aspect, in a case where the first region of interest is measured, the first region of interest and the second region of interest, which are to be compared, can be displayed by applying the same display aspect.

According to an image processing device according to a ninth aspect, in the image processing device according to the eighth aspect, the one or more processors may be configured to set at least any one of brightness, a window level, or a window width as the first display condition.

According to this aspect, it is possible to display the first region of interest and the second region of interest in which at least any one of the brightness, the window level, or the window width is adjusted.

According to an image processing device according to a tenth aspect, in the image processing device according to any one of the first aspect to the ninth aspect, the one or more processors may be configured to display at least any one of the first region of interest or a measurement result of the first region of interest on a display device in a case where an input for selecting the first region of interest in the first image is acquired.

In this aspect, an input for selecting the first region of interest in the first image corresponding to an operation of the user may be acquired.

According to an image processing device according to an eleventh aspect, in the image processing device according to any one of the first aspect to the ninth aspect, the one or more processors may be configured to display at least one of the first region of interest or a measurement result of the first region of interest on a display device and to display the measurement information on the display device in a case where an input for selecting the first region of interest in the first image is acquired.

According to this aspect, it is possible to contribute to the improvement of the user convenience.

In this aspect, an input for selecting the first region of interest in the first image corresponding to an operation of the user may be acquired.

According to an image processing device according to a twelfth aspect, in the image processing device according to the eleventh aspect, the one or more processors may be configured to apply different display aspects to at least any one of the first region of interest or the measurement result of the first region of interest, and the measurement information.

According to this aspect, the information belonging to the first image and the information belonging to the second image can be easily distinguished.

In this aspect, an input for selecting the first region of interest in the first image corresponding to an operation of the user may be acquired.

According to an image processing device according to a thirteenth aspect, in the image processing device according to the eleventh aspect, the one or more processors may be configured to display at least any one of the second image or a measurement result of the second region of interest as the measurement information on the display device in a case where an input for selecting the first region of interest in the first image is acquired.

In this aspect, an input for selecting the first region of interest in the first image corresponding to an operation of the user may be acquired.

According to an image processing device according to a fourteenth aspect, in the image processing device according to any one of the first aspect to the thirteenth aspect, the one or more processors may be configured to: receive, as the plurality of first images, a plurality of first medical images generated by imaging a subject; designate, as the first region of interest, an anatomical structure of the subject in the plurality of first medical images; and acquire, as the measurement information, measurement information obtained by measuring the anatomical structure of the subject which is the second region of interest corresponding to the first region of interest for a plurality of second medical images generated by imaging the subject.

According to this aspect, the accurate comparison between the measurement result of the first region of interest in the first medical image and the measurement result of the second region of interest in the second medical image can be realized.

According to an image processing device according to a fifteenth aspect, in the image processing device according to the fourteenth aspect, the one or more processors may be configured to display a measurement result of the anatomical structure on a display device as a measurement result of the first region of interest in the first image.

According to this aspect, the accurate comparison is realized on the measurement results of the first region of interest and the second region of interest to which the anatomical structure is applied.

An operation method of an image processing device according to a sixteenth aspect of the present disclosure is an operation method of an image processing device including one or more processors and one or more memories that store a program to be executed by the one or more processors, the method comprises: via the one or more processors, receiving a plurality of first images that are generated by imaging a subject and to which each of a plurality of first image conditions different from each other is applied; designating a first region of interest in the plurality of first images; acquiring, for a plurality of second images that are generated by imaging the subject before capturing the first image, measurement information on the second image having a second region of interest corresponding to the first region of interest; specifying the first image included in a range of the measurement information from the plurality of first images; and measuring the first region of interest in the specified first image.

According to the operation method of an image processing device according to the sixteenth aspect of the present disclosure, it is possible to obtain the same actions and effects as those of the image processing device according to the first aspect of the present disclosure.

In the operation method of an image processing device according to the sixteenth aspect, the same matters as those specified in the second aspect to the fifteenth aspect can be appropriately combined. In this case, the component responsible for the processing or the function specified in the image processing device can be grasped as a component of the operation method of the image processing device responsible for the corresponding processing or function.

A program according to a seventeenth aspect of the present disclosure is a program for causing a computer to implement: a function of receiving a plurality of first images that are generated by imaging a subject and to which each of a plurality of first image conditions different from each other is applied; a function of designating a first region of interest in the plurality of first images; a function of acquiring, for a plurality of second images that are generated by imaging the subject before capturing the first image, measurement information on the second image having a second region of interest corresponding to the first region of interest; a function of specifying the first image included in a range of the measurement information from the plurality of first images; and a function of measuring the first region of interest in the specified first image.

According to the program according to the seventeenth aspect of the present disclosure, the same actions and effects as those of the image processing device according to the first aspect of the present disclosure can be obtained.

In the program according to the seventeenth aspect, the same matters as those specified in the second aspect to the fifteenth aspect can be appropriately combined. In this case, the component responsible for the processing or the function specified in the image processing device can be grasped as a component of the program responsible for the corresponding processing or function.

According to the present invention, based on the measurement information on the second image having the second region of interest corresponding to the first region of interest designated in the first image, the first image included in the range of measurement information is specified, and the first region of interest in the specified first image is measured. Accordingly, the accurate comparison between the measurement result of the first region of interest in the first image and the measurement result of the second region of interest in the second image is realized.

### BRIEF DESCRIPTION OF THE DRAWINGS

FIG. 1 is an overall configuration diagram of a medical image processing system.

FIG. 2 is a schematic diagram of an image processing method according to an embodiment.

FIG. 3 is a schematic diagram illustrating a procedure of an image processing method according to a first embodiment.

FIG. 4 is a functional block diagram illustrating an electric configuration of an image processing device according to the first embodiment.

FIG. 5 is a block diagram schematically illustrating an example of a hardware configuration of the image processing device according to the first embodiment.

FIG. 6 is a table illustrating priorities given to image conditions.

FIG. 7 is a table illustrating points given to the image conditions.

FIG. 8 is a schematic diagram illustrating a configuration example of a user interface.

FIG. 9 is a schematic diagram illustrating a procedure of an image processing method according to a second embodiment.

FIG. 10 is a functional block diagram illustrating an electric configuration of an image processing device according to the second embodiment.

FIG. 11 is a block diagram schematically illustrating an example of a hardware configuration of the image processing device according to the second embodiment.

FIG. 12 is a schematic diagram illustrating a procedure of an image processing method according to a third embodiment.

FIG. 13 is a functional block diagram illustrating an electric configuration of an image processing device according to the third embodiment.

FIG. 14 is a block diagram schematically illustrating an example of a hardware configuration of an image processing system according to the third embodiment.

### DESCRIPTION OF THE PREFERRED EMBODIMENTS

Hereinafter, a preferred embodiment of the present invention will be described in accordance with the accompanying drawings. In the present specification, the same components are designated by the same reference numerals, and duplicate description thereof will be omitted as appropriate.

### Configuration Example of Medical Image Processing System

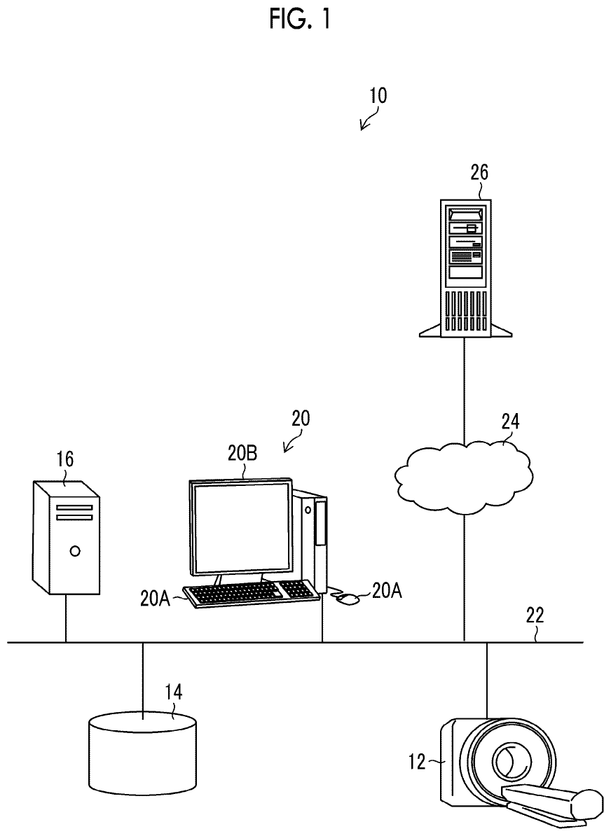

FIG. 1 is an overall configuration diagram of a medical image processing system. As illustrated in FIG. 1, a medical image processing system 10 is a system that captures an image of a person to be examined as a subject and that automatically executes image processing which is for assisting in diagnosis of a medical image for the captured image and which is image processing for the medical image. It should be noted that the person to be examined can be referred to as a subject or the like.

As illustrated in FIG. 1, the medical image processing system 10 comprises a medical image examination device 12, a medical image database 14, an image processing device 16, a user terminal 20, and a cloud server 26. The medical image examination device 12, the medical image database 14, the image processing device 16, and the user terminal 20 are provided in a medical institution, such as a hospital, and are connected to each other via an in-hospital network 22 so as to be able to transmit and receive data. As the in-hospital network 22, a local area network (LAN) can be applied. The in-hospital network 22 maybe wired or wireless.

The in-hospital network 22 is connected to the Internet 24 via a router (not illustrated). The in-hospital network 22 and the cloud server 26 are connected to each other via the Internet 24 so as to be able to freely transmit and receive data.

The medical image examination device 12 is an imaging device that images an examination target part of the person to be examined and generates a medical image. Examples of the medical image examination device 12 include an X-ray imaging device, a CT device, an MRI device, a PET device, an ultrasound device, and a CR device using a flat X-ray detector.

It should be noted that CT is an abbreviation for computed tomography. MRI is an abbreviation for magnetic resonance imaging. PET is an abbreviation for positron emission tomography. CR is an abbreviation for computed radiography.

The medical image database 14 is a database for managing medical images captured by using the medical image examination device 12. As the medical image database 14, a computer comprising a high-capacity storage device can be applied. Software for providing functions of a database management system is incorporated into the computer.

The medical image database 14 maybe configured as a part of an image storage communication system that stores and manages the medical images. The image storage communication system is referred to as PACS, using an abbreviation for picture archiving and communication systems.

As a format of the medical image, a DICOM standard can be applied. DICOM tag information prescribed by the DICOM standard may be added to the medical image. It should be noted that the term “image” in the present specification can include the meaning of image data, which is a signal representing an image, in addition to the meaning of an image itself such as a photograph. It should be noted that the DICOM is an abbreviation for digital imaging and communications in medicine.

The image processing device 16 executes prescribed image processing on a medical image generated by imaging the person to be examined using the medical image examination device 12 and on the medical image stored in the medical image database 14. As the image processing device 16, a computer is applied. The computer may be a personal computer, a workstation, or a tablet terminal. The computer may be a virtual machine.

The user terminal 20 is a terminal device used by a user, such as a doctor, and, for example, a known image viewer for image interpretation is applied. The user terminal 20 maybe a personal computer, a workstation, or a tablet terminal.

The user terminal 20 comprises an input device 20A and a display device 20B. The medical image captured by the medical image examination device 12 and a result of the image processing are displayed on the display device 20B. In addition, a result of the image processing, which is executed by the image processing device 16 and the cloud server 26, is displayed on the display device 20B. The user can input an instruction to the medical image processing system 10 by using the input device 20A.

As the cloud server 26, for example, a server computer, a personal computer, or a workstation can be applied. The cloud server 26 performs image processing including at least any one of lesion extraction or disease name determination processing on the medical image of the person to be examined. The cloud server 26 can be accessed from the in-hospital networks 22 of a plurality of hospitals via the Internet 24.

### Outline of Image Processing Method according to Embodiment

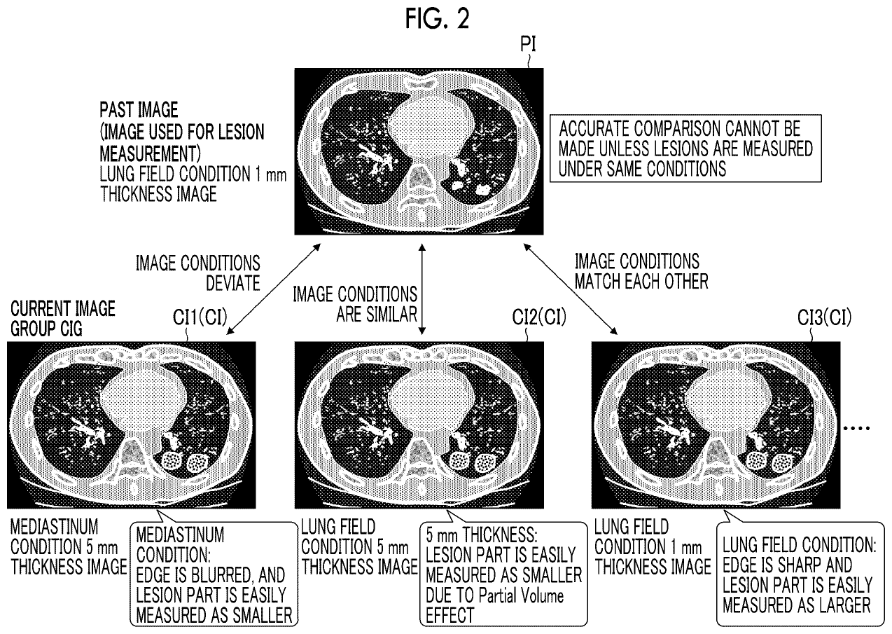

FIG. 2 is a schematic diagram of an image processing method according to the embodiment. The image processing method according to the embodiment is realized by the image processing device 16 illustrated in FIG. 1. The image processing method according to the embodiment includes acquiring, in a case where measurement of a lesion part in a current image is performed, measurement information of a previous examination of the lesion part designated in the current image, and specifying a current image having image conditions that are the same as or similar to those of the past image used in the previous measurement from the current image group including the plurality of current images based on the acquired measurement information.

Specifically, a current image group CIG including a plurality of current images CI illustrated in FIG. 2 is acquired. In FIG. 2, a current image CI1, a current image CI2, and a current image CI3 are illustrated as the plurality of current images CI. The current image group CIG may be acquired from the medical image examination device 12 illustrated in FIG. 1 or may be acquired from the medical image database 14. It should be noted that the image processing method described in the embodiment is an example of an operation method of the image processing device.

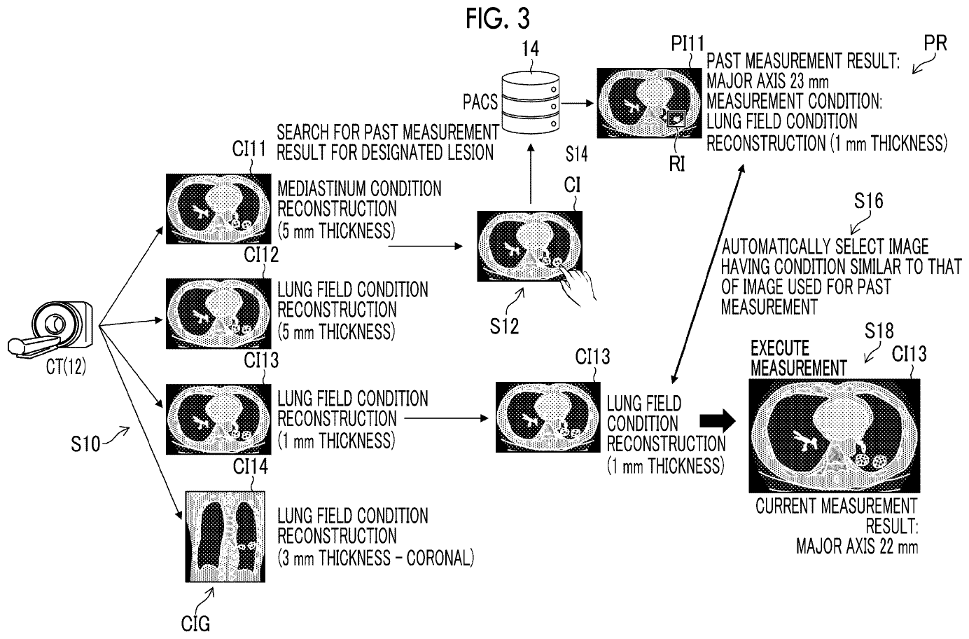

FIG. 2 illustrates a CT image, which is a two-dimensional slice image representing an axial cross section reconstructed from three-dimensional volume data, as a medical image. For the medical image illustrated in FIG. 3 or the like, a two-dimensional slice image is also illustrated as an example.

In addition, FIG. 2 illustrates reconstruction conditions and slice thicknesses as image conditions of the plurality of current images CI. A reconstruction condition of the current image CI1 is a mediastinum condition, and a slice thickness of the current image CI1 is 5 mm. A reconstruction condition of the current image CI2 is a lung field condition, and a slice thickness of the current image CI2 is 5 mm. A reconstruction condition of the current image CI3 is the lung field condition, and a slice thickness of the current image CI3 is 1 mm.

Next, for a lesion part designated in any current image CI, a past image PI used for lesion measurement at the time of the previous examination is acquired. The past image PI is acquired from the medical image database 14 illustrated in FIG. 1. A reconstruction condition of the past image PI illustrated in FIG. 2 is the lung field condition, and a slice thickness of the past image PI is 1 mm.

Next, the current image CI having the same image conditions as the past image PI or having image conditions similar to the image conditions of the past image PI is specified. In the example illustrated in FIG. 2, the current image CI3 having the same image conditions as those of the past image PI is specified.

The current image CI1 is different from the past image PI in reconstruction condition and slice thickness. The current image CI1 is a current image CI whose image conditions do not match those of the past image PI and whose image conditions deviate from those of the past image PI.

The current image CI2 has the same reconstruction conditions as the past image PI, but the slice thickness is different. The current image CI2 is a current image CI whose image conditions partially match those of the past image PI and whose image conditions are similar to those of the past image PI.

For example, in a case where the reconstruction condition is the mediastinum condition, the edge is blurred and unclear, and the lesion part tends to be easily measured as smaller. On the other hand, in a case where the reconstruction condition is the lung field condition, the edge is sharp and clear, and the lesion part tends to be easily measured as larger.

In addition, in a case where the slice thickness is 5 mm, it is affected by the partial volume effect, and the lesion part tends to be easily measured as smaller than a case where the slice thickness is 1 mm. Then, in a case where the lesion part is not measured using the current image CI to which an image condition that is the same as or similar to the past image PI used for comparison of the measurement results is applied, the accurate comparison between the current lesion part and the past lesion part becomes difficult. The image processing method according to the embodiment solves the above problems, and realizes preferable comparison between the measurement result of the lesion part of the current image CI and the measurement result of the lesion part of the past image.

### Procedure of Image Processing Method according to First Embodiment

FIG. 3 is a schematic diagram illustrating a procedure of an image processing method according to a first embodiment. In a current image group reception step S10, the image processing device 16 illustrated in FIG. 1 receives the current image group CIG including the plurality of current images CI having different image conditions. FIG. 3 illustrates a current image CI11, a current image CI12, a current image CI13, and a current image CI14, which are generated by performing the series of single imaging on the same patient using the CT device which is the medical image examination device 12, as a plurality of current images CI.

The image processing device 16 mayreceive the plurality of current images CI from the medical image examination device 12 such as the CT device illustrated in FIG. 3, or may receive the plurality of current images CI from the medical image database 14.

The current image CI11 illustrated in FIG. 3 is an axial cross section image, the reconstruction condition is a mediastinum condition, and the slice thickness is 5 mm. The current image CI12 is an axial cross section image, the reconstruction condition is a lung field condition, and the slice thickness is 5 mm. The current image CI13 is an axial cross section image, the reconstruction condition is a lung field condition, and the slice thickness is 1 mm. The current image CI14 is a coronal cross section image, the reconstruction condition is a mediastinum condition, and the slice thickness is 3 mm.

In a lesion part designation step S12, the image processing device 16 designates a lesion part RI for any current image CI among the plurality of current images CI received in the current image group reception step S10. For example, any current image CI is displayed on the display device 20B of the user terminal 20, and the user operates the input device 20A of the user terminal 20 to designate the lesion part RI for any current image CI displayed on the display device 20B.

In a measurement information acquisition step S14, the image processing device 16 acquires measurement information obtained by measuring the lesion part of the past image PI for the lesion part RI designated in any current image CI. That is, the image processing device 16 extracts a medical image whose patient information and examination information match from the medical images stored in the medical image database 14, and searches for the past measurement result or the past image used for the past measurement for the lesion part RI designated in any current image CI from the extracted medical image, as the past measurement information. The patient information includes identification information of the patient such as the name of the patient. The examination information includes identification information of the examination, such as an examination ID, and information on a modality used for the examination. It should be noted that ID is an abbreviation for identification.

FIG. 3 illustrates a past image PI11 and a measurement result PR of the past image PI11 as past measurement information. The past measurement information may be the past image PI11, may be the measurement result PR of the past image PI11, or may be the past image PI11 and the measurement result PR of the past image PI11.

In a current image specifying step S16, the current image CI having the same image condition as the image condition of the past image PI11 acquired as the past measurement information or having an image condition similar to the image condition of the past image PI11 acquired as the past measurement information is specified. That is, the image processing device 16 specifies the current image CI included in a range of the past measurement information from the current image CI11, the current image CI12, the current image CI13, and the current image CI14.

The reconstruction condition of the past image PI11 is the lung field condition, and the slice thickness is 1 mm. The reconstruction condition of the current image CI13 is the lung field condition, the slice thickness is 1 mm, and the image condition of the current image CI13 matches the image condition of the past image PI11. Therefore, in the current image specifying step S16, the current image CI13 whose image conditions match the past image PI11 used for the past measurement is specified and automatically selected.

FIG. 3 illustrates the reconstruction condition and the slice thickness as the image condition. The image condition may include a pixel spacing, an imaging protocol, and the like. That is, as the image condition, at least any one of the reconstruction condition, the slice thickness, the pixel spacing, or the imaging protocol can be applied. It should be noted that the slice thickness described in the embodiment is an example of a slice thickness representing a thickness of a tomographic layer in a tomographic image, and the pixel spacing described in the embodiment is an example of spatial resolution.

In a current image measurement step S18, the image processing device 16 measures the lesion part RI on the current image CI13 specified in the current image specifying step S16. The image processing device 16 maydisplay the measurement result of the lesion part RI on the display device 20B of the user terminal 20. Examples of a measurement target of the lesion part RI include a major axis, a minor axis, a volume, an average CT value, and an average signal value of the lesion part RI.

It should be noted that the current image CI described in the embodiment is an example of a first image and is an example of a first medical image. The past image PI described in the embodiment is an example of a second image generated by imaging a subject before the first image is captured, and is an example of the second image having a second region of interest corresponding to the first region of interest. In addition, the past image PI described in the embodiment is an example of a second medical image.

In addition, the lesion part RI designated in any current image CI described in the embodiment is an example of a first region of interest. The lesion part of the past image PI described in the embodiment is an example of a second region of interest. The image condition of the current image described in the embodiment is an example of a first image condition. The past measurement information described in the embodiment is an example of measurement information.

### Electric Configuration Example of Image Processing Device according to First Embodiment

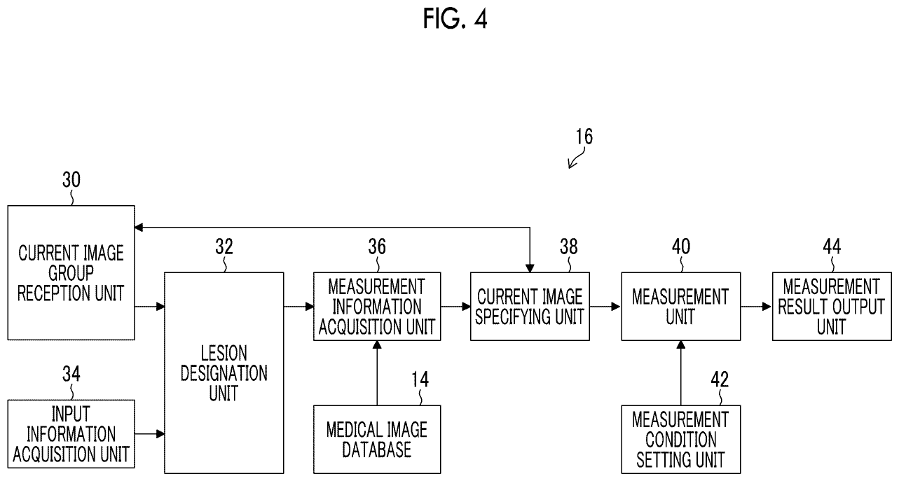

FIG. 4 is a functional block diagram illustrating an electric configuration of an image processing device according to the first embodiment. The image processing device 16 illustrated in FIG. 4 comprises a current image group reception unit 30. The current image group reception unit 30 executes the current image group reception step S10 illustrated in FIG. 3 to receive the current image group CIG including the plurality of current images.

The image processing device 16 comprises a lesion designation unit 32 and an input information acquisition unit 34. The lesion designation unit 32 executes the lesion part designation step S12 to designate the lesion part RI using any current image CI included in the plurality of current images CI. Examples of the lesion part include a tumor, a mass, and an ulcer. The lesion designation unit 32 maydesignate an anatomical structure, such as a bone, a nerve, and a blood vessel, as the region of interest.

The input information acquisition unit 34 acquires input information to the image processing device 16. For example, the input information acquisition unit 34 can acquire designation information of the lesion part RI of the user for any current image CI. The lesion designation unit 32 can designate the lesion part RI for any current image CI based on the designation information of the lesion part RI of the user for any current image CI acquired by the input information acquisition unit 34.

The image processing device 16 comprises a measurement information acquisition unit 36. The measurement information acquisition unit 36 executes the measurement information acquisition step S14, searches for the medical image database 14, and acquires the past measurement information of the lesion part RI designated for the current image CI from the medical image database 14. The past measurement information may be the past image PI obtained by measuring the lesion part RI or may be the measurement result PR of the past lesion part RI.

The image processing device 16 comprises a current image specifying unit 38. The current image specifying unit 38 executes the current image specifying step S16, specifies image conditions of the past image PI corresponding to the past measurement information acquired by the measurement information acquisition unit 36, and specifies the current image CI having the same image condition as the image condition of the specified past image PI or having an image condition similar to the image condition of the specified past image PI. The current image specifying unit 38 specifies the current image CI having the image condition satisfying the above from the plurality of current images CI acquired in the current image group reception step S10.

The image processing device 16 comprises a measurement unit 40 and a measurement condition setting unit 42. The measurement unit 40 applies a measurement condition set by the measurement condition setting unit 42 and measures the lesion part RI of the current image CI specified by the current image specifying unit 38. The measurement of the lesion part RI in the current image CI may be performed by extracting a pixel included in the lesion part RI or may be performed based on the pixel of a region designated by the user. The measurement condition includes measurement parameters for the lesion part RI, such as the major axis, the minor axis, and the area.

The image processing device 16 comprises a measurement result output unit 44. The measurement result output unit 44 outputs a measurement result of the lesion part RI of the current image CI measured by using the measurement unit 40. Examples of the output of the measurement result include the display of the measurement result on the display device 20B illustrated in FIG. 1. Another example of the output of the measurement result includes the printing of the measurement result using a printing device.

### Hardware Configuration Example of Image Processing Device according to First Embodiment

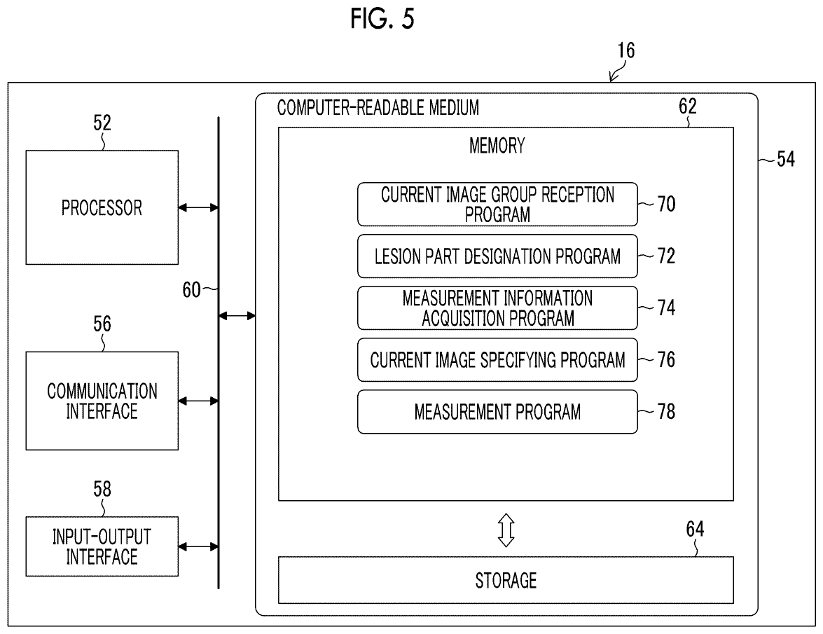

FIG. 5 is a block diagram schematically illustrating an example of a hardware configuration of the image processing device according to the first embodiment. The image processing device 16 comprises one or more processors 52 and one or more memories 62. The image processing device 16 comprises a communication interface 56 and an input-output interface 58.

The processor 52 executes various programs stored in the memory 62 of the computer-readable medium 54 to implement various functions of the image processing device 16. The processor 52 includes a central processing unit (CPU). The processor 52 mayinclude a graphics processing unit (GPU). The processor 52 is connected to the computer-readable medium 54, the communication interface 56, and the input-output interface 58 via the bus 60.

The computer-readable medium 54 includes the memory 62 which is a main storage device and a storage 64 which is an auxiliary storage device. A semiconductor memory, a hard disk apparatus, a solid state drive apparatus, and the like can be applied to the computer-readable medium 54. Any combination of a plurality of devices can be applied to the computer-readable medium 54.

It should be noted that the hard disk apparatus can be referred to as HDD that is an abbreviation for hard disk drive in English notation. The solid state drive apparatus can be referred to as SSD that is an abbreviation for solid state drive in English notation.

The memory 62 of the computer-readable medium 54 stores a current image group reception program 70, a lesion part designation program 72, a measurement information acquisition program 74, a current image specifying program 76, and a measurement program 78.

The current image group reception program 70 is applied to the current image group reception unit 30 illustrated in FIG. 4 and implements a current image group reception function. The lesion part designation program 72 is applied to the lesion designation unit 32 and implements a lesion part designation function. The measurement information acquisition program 74 is applied to the measurement information acquisition unit 36 and implements a measurement information acquisition function. The current image specifying program 76 is applied to the current image specifying unit 38 and implements current image specification. The measurement program 78 is applied to the measurement unit 40 and implements a measurement function.

Various programs stored in the computer-readable medium 54 include one or more commands. The computer-readable medium 54 stores various types of data, various parameters, and the like. It should be noted that the term “program” is synonymous with the term “software”.

A hardware structure of the processor 52 is various processors as described below. The various processors include a central processing unit (CPU) that is a general-purpose processor actioning as various functional units by executing software (program), a graphics processing unit (GPU) that is a processor specially designed for image processing, a programmable logic device (PLD) such as a field programmable gate array (FPGA) that is a processor having a circuit configuration changeable after manufacturing, and a dedicated electric circuit or the like such as an application specific integrated circuit (ASIC) that is a processor having a circuit configuration dedicatedly designed to execute a specific type of processing.

One processing unit may be configured by one processor among these various processors, or may be configured by two or more same or different kinds of processors (for example, a combination of a plurality of FPGAs, a combination of the CPU and the FPGA, or a combination of the CPU and GPU). In addition, a plurality of functional units may be formed of one processor. As an example of configuring the plurality of functional units with one processor, first, as represented by a computer such as a client or a server, a form of configuring one processor with a combination of one or more CPUs and software and causing the processor to act as the plurality of functional units is present. Second, as represented by a system on chip (SoC) or the like, a form of using a processor that implements the function of the entire system including the plurality of functional units using one integrated circuit (IC) chip is present. Accordingly, various functional units are configured using one or more of the various processors as a hardware structure.

Furthermore, the hardware structure of the various processors is more specifically an electric circuit (circuitry) in which circuit elements such as semiconductor elements are combined.

The memory 62 stores the command to be executed by the processor 52. The memory 62 includes a random access memory (RAM) and a read only memory (ROM) which are not illustrated. The processor 52 uses the RAM as a work region to execute software using various programs and parameters including an image processing program including the current image group reception program 70 stored in the ROM, and executes various types of processing of the image processing device 16 using the parameters stored in the ROM.

The user terminal 20 illustrated in FIG. 1 mayimplement various functions of the image processing device 16. That is, the user terminal 20 maycomprise various processing units such as the current image group reception unit 30 illustrated in FIG. 4, and may execute various steps such as the current image group reception step S10 illustrated in FIG. 3. In addition, the cloud server 26 illustrated in FIG. 1 mayimplement various functions of the image processing device 16.

### Specific Example of Specifying Current Image for Measuring Lesion Part

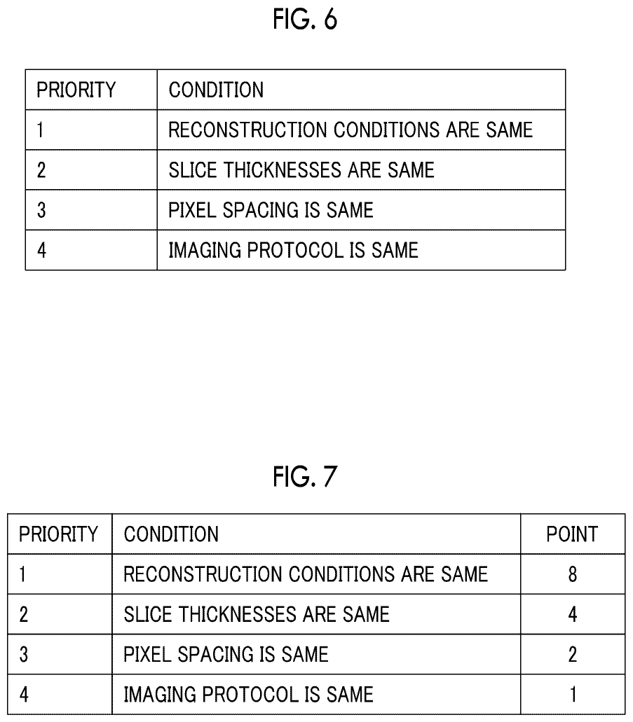

FIG. 6 is a table illustrating priorities given to the image conditions. In FIG. 6, as the image condition, the reconstruction condition, the slice thickness, the pixel spacing, and the imaging protocol are illustrated. The imaging protocol can include distinction between contrast and non-contrast. The imaging protocol can include a dose.

In the current image specifying step S16 illustrated in FIG. 3, the number of matches of the image conditions is calculated for all the current images CI included in the current image group CIG. The current image CI having the maximum number of matches is specified as the current image CI having the similar image condition. In a case where there are a plurality of current images CI having the same number of matches, the current image CI that matches the image condition with a high priority is specified as the current image CI having a similar image condition.

FIG. 7 is a table illustrating points given to the image conditions. The point may be defined for each image condition. In the current image specifying step S16, the sum of the points of matching image conditions is calculated for all the current images CI included in the current image group CIG. The current image CI having the maximum point is specified as the current image CI having the similar image condition. In a case where there are a plurality of current images CI having the same sum of the points, the current image CI that matches the image condition with a high priority is specified as the current image CI having a similar image condition.

It should be noted that the sum of the points described in the embodiment is an example of a similarity indicator representing similarity to the second image condition. The image condition of the past image PI described in the embodiment is an example of the second image condition.

### Configuration Example of User Interface

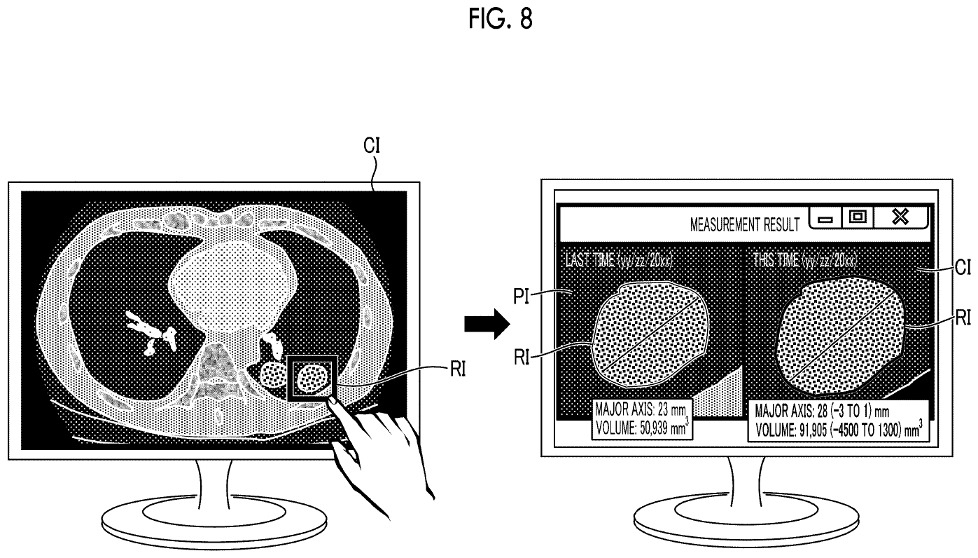

FIG. 8 is a schematic diagram illustrating a configuration example of the user interface. In a case where the user executes a click operation on the lesion part RI of the current image CI displayed on the display device 20B illustrated in FIG. 2, a transition may be made from a screen in which the current image CI is displayed to a screen in which the past image PI and the measurement result in the past image PI are popped up and the measurement result in the current image CI are popped up for the current image CI.

In addition, as illustrated in FIG. 8, on the screen after the transition, the lesion part RI is enlarged and displayed, and text information representing the measurement result may be superimposed and displayed at a position in the vicinity of the lesion part RI and not overlapping the lesion part RI. Furthermore, the lesion part RI of the past image PI and the lesion part RI of the current image CI may be distinguished by using colors. In addition, the measurement result of the past image PI and the measurement result of the current image CI may be distinguished by using colors.

The same color may be applied to the lesion part RI of the past image PI and the measurement result of the past image PI. The same color may be applied to the lesion part RI of the current image CI and the measurement result of the current image CI. Text information representing the past image PI and text information representing the examination date may be superimposed and displayed on the past image PI. Text information representing the current image CI and text information representing the examination date may be superimposed and displayed on the current image CI. That is, since different display aspects are applied to the past image PI and information related to the past image PI and the current image CI and information related to the current image CI, both can be visually distinguished.

### Actions and Effects of First Embodiment

The image processing method and the image processing device according to the first embodiment can obtain the following actions and effects.

[1]

In a case where the lesion part RI designated in the current image CI is measured, the past measurement information of the lesion part RI as a measurement target is acquired, the current image CI within a range of the past measurement information is specified, and the lesion part RI in the specified current image CI is measured. Accordingly, it is possible to accurately compare the lesion part RI of the current image CI with the lesion part RI of the past image PI.

[2]

The current image CI within the range of the past measurement information includes the current image CI having the same image conditions as the image conditions of the past image PI in which the past measurement is performed, and the current image CI having the image conditions similar to the image conditions of the past image PI in which the past measurement is performed. Accordingly, even in a case where there is no current image CI having the same image conditions as the image conditions of the past image PI in which the past measurement is performed, it is possible to specify the current image CI in which the measurement of the lesion part RI is performed.

[3]

Image conditions similar to the image conditions of the past image PI are determined based on the similarity of the reconstruction conditions, the similarity of the slice thicknesses, the similarity of the pixel spacing, and the similarity of the imaging protocol. Accordingly, it is possible to specify the current image CI suitable for measuring the lesion part RI.

[4]

The similarity of the image conditions is determined based on the prescribed priority. Accordingly, the accurate determination of the similarity of the image conditions is performed.

### Procedure of Image Processing Method according to Second Embodiment

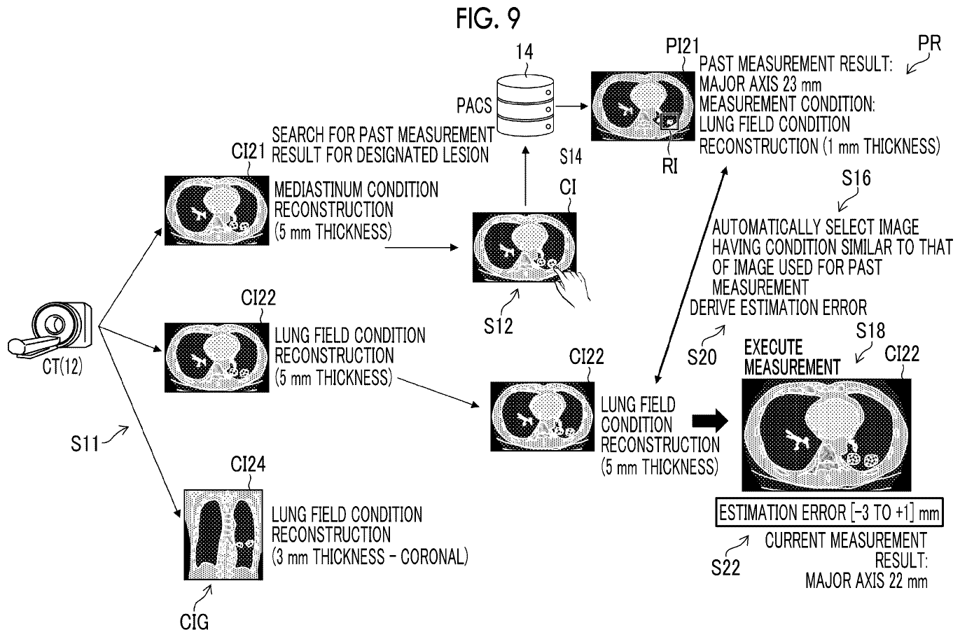

FIG. 9 is a schematic diagram illustrating a procedure of an image processing method according to a second embodiment. The image processing method according to the second embodiment includes a current image group reception step S11 instead of the current image group reception step S10 illustrated in FIG. 3. In addition, the image processing method according to the second embodiment is different from the image processing method according to the first embodiment in that an estimation error derivation step S20 and an estimation error output step S22 are added.

In the current image group reception step S11 illustrated in FIG. 9, a current image group CIG including a current image CI21, a current image CI22, and a current image CI24 is received. In the lesion part designation step S12, as in the lesion part designation step S12 illustrated in FIG. 3, the lesion part RI is designated for any current image CI included in the current image group CIG.

In the measurement information acquisition step S14, as in the measurement information acquisition step S14 illustrated in FIG. 3, the past measurement information of the designated lesion part RI is acquired. FIG. 9 illustrates an example in which a past image PI21 and a past measurement result are acquired as the past measurement information.

In the current image specifying step S16, since there is no current image CI having the same image conditions as the image conditions of the past image PI21, the current image CI22 having the image conditions similar to the image conditions of the past image PI21 is specified.

Here, the past image PI21 and the current image CI22 have the same reconstruction condition but have different slice thicknesses. An error may occur between the measurement result of the lesion part RI of the current image CI22 having image conditions different from those of the past image PI21 and the measurement result of the lesion part RI of the past image PI21.

In the estimation error derivation step S20, an estimation error between the measurement result of the lesion part RI in the past image PI21 and the measurement result of the lesion part RI in the current image CI22 is derived. In the estimation error derivation step S20, an estimation error database in which the estimation error for each combination of the image conditions is stored is used.

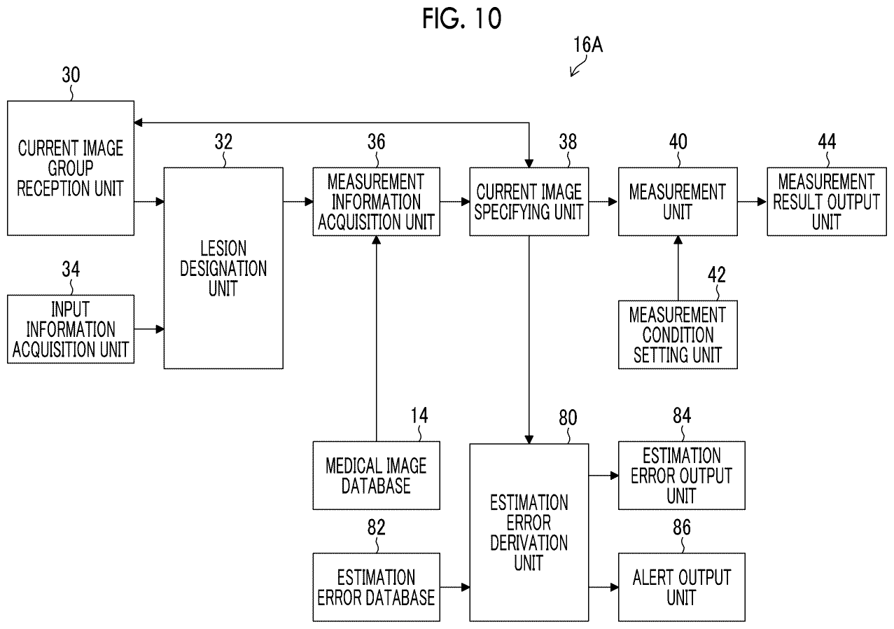

In the estimation error database, for the same lesion part RI, an error of the measurement result in which the measurement is performed under different image conditions and a combination of the image conditions are associated and stored. The estimation error database may be generated for each type of the lesion part. It should be noted that the estimation error database is illustrated in FIG. 10 by using reference numeral 82.

In the estimation error output step S22, the estimation error derived in the estimation error derivation step S20 is output. FIG. 9 illustrates the display of text information on the display device as the output of the estimation error. FIG. 9 illustrates an example in which −3 mm or more and +1 mm or less are displayed as a range of the estimation error.

In the example illustrated in FIG. 9, while the slice thickness of the past image PI21 is 1 mm, the slice thickness of the current image CI22 is 5 mm, and the current image CI22 is likely to be measured as smaller than the past image PI21. Therefore, the range of the estimation error is adjusted such that a numerical range on the negative side is larger than a numerical range on the positive side.

In a case where the estimation error exceeds a prescribed range, an alert output step of outputting an alert may be executed. As an output form of the alert, sound, voice, display of text information, or the like can be applied. For the output of the alert, a plurality of output forms such as a combination of the voice and the text information may be combined. It should be noted that the display device described in the embodiment is an example of a display device.

### Electric Configuration Example of Image Processing Device according to Second Embodiment

FIG. 10 is a functional block diagram illustrating an electric configuration of an image processing device according to the second embodiment. The image processing device 16A illustrated in FIG. 10 is different from the image processing device 16 illustrated in FIG. 4 in that an estimation error derivation unit 80 and an estimation error output unit 84 are added. An alert output unit 86 maybe added to the image processing device 16A.

The estimation error derivation unit 80 executes the estimation error derivation step S20 illustrated in FIG. 9 and derives the estimation error of the measurement of the lesion part RI in the current image CI to which the image condition different from that of the past image PI is applied with reference to the estimation error database 82. The estimation error database 82 maybe provided in the image processing device 16A or may be an external device of the image processing device 16A.

The estimation error output unit 84 executes the estimation error output step S22 and outputs the estimation error derived by the estimation error derivation unit 80. The estimation error output unit 84 maydisplay the estimation error on the display device.

The alert output unit 86 outputs an alert in a case where the estimation error derived by the estimation error derivation unit 80 exceeds the prescribed range. An alert output condition setting unit that sets an output condition of the alert for the alert output unit 86 maybe provided.

### Hardware Configuration Example of Image Processing Device according to Second Embodiment

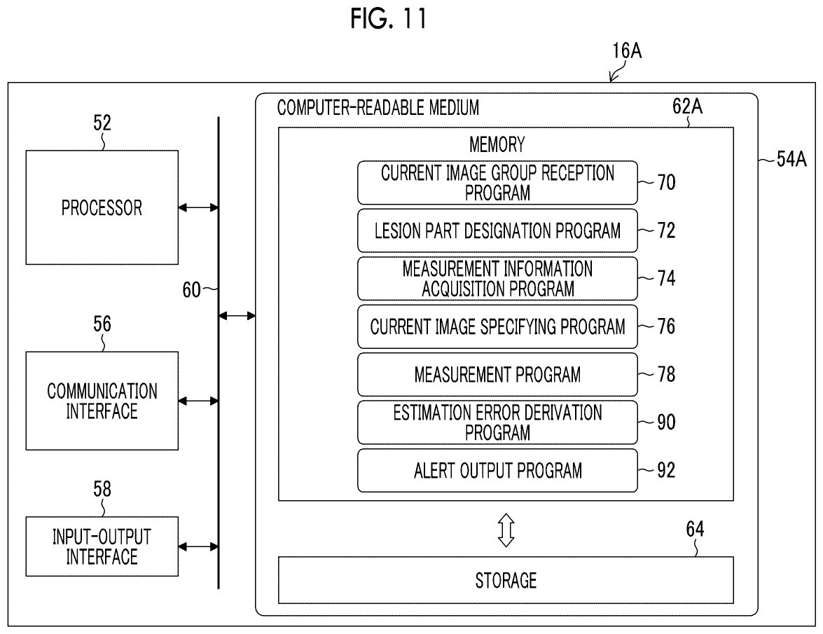

FIG. 11 is a block diagram schematically illustrating an example of a hardware configuration of the image processing device according to the second embodiment. The image processing device 16A illustrated in FIG. 11 comprises a computer-readable medium 54A instead of the computer-readable medium 54 of the image processing device 16 illustrated in FIG. 5. The computer-readable medium 54A comprises a memory 62A instead of the memory 62 illustrated in FIG. 5.

Various programs, such as a current image group reception program 70 stored in the memory 62 illustrated in FIG. 5, are stored in the memory 62A, and an estimation error derivation program 90 is added to the memory 62A. An alert output program 92 maybe added to the memory 62A.

The estimation error derivation program 90 is applied to the estimation error derivation unit 80 illustrated in FIG. 9 and implements an estimation error derivation function. The alert output program 92 is applied to the alert output unit 86 and implements an alert output function.

### Actions and Effects of Second Embodiment

The image processing method and the image processing device according to the second embodiment can obtain the following actions and effects.

[1]

In a case where the current image CI whose image condition is not the same as that of the past image PI and whose image condition is similar to the image condition of the past image PI is specified, an estimation error with respect to the measurement result of the current image CI is derived and output. Accordingly, the accurate comparison of the measurement results between the current image CI and the past image PI is realized.

[2]

The estimation error is derived in advance by using the estimation error database in which different image conditions are applied and a difference between measurement results obtained by measuring the lesion part is stored in association with a combination of the image conditions. Accordingly, the derivation of the estimation error with high reliability is realized.

[3]

In a case where the estimation error exceeds the prescribed range, an alert indicating that fact is output. Accordingly, the user can grasp a case where it is difficult to accurately compare the measurement results between the current image CI and the past image PI.

### Procedure of Image Processing Method according to Third Embodiment

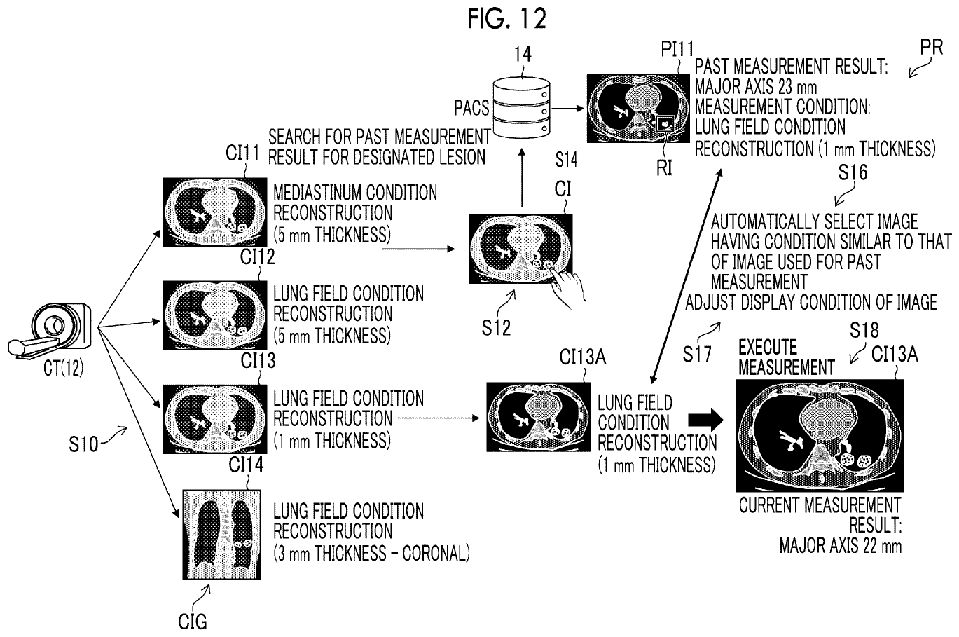

FIG. 12 is a schematic diagram illustrating a procedure of an image processing method according to a third embodiment. The image processing method according to the third embodiment is different from the image processing method according to the first embodiment in that a display condition adjustment step S17 is added.

The display condition adjustment step S17 is executed after the current image specifying step S16, and the display condition of the current image CI13 specified in the current image specifying step S16 is adjusted in accordance with the display condition of the past image PI11.

In the display condition adjustment step S17, the display condition of the current image CI13 is adjusted by using the display condition of the past image PI acquired in a case where the past measurement information is acquired in the measurement information acquisition step S14.

In the example illustrated in FIG. 12, in the current image specifying step S16, the brightness of the past image PI11 is darker than the specified current image CI13, and the current image CI13A in which the brightness is adjusted in accordance with the past image PI11 is displayed.

In the current image measurement step S18, the measurement of the lesion part RI is performed by using the current image CI13A in which the brightness is adjusted in accordance with the past image PI11. As the display condition to be adjusted, at least any one of a window level representing a reference of a CT value desired to be represented on the image or a window width representing a range of the CT value desired to be represented on the image may be applied.

It should be noted that the display condition of the current image CI13 described in the embodiment is an example of a first display condition. The display condition of the past image PI11 described in the embodiment is an example of a second display condition.

### Electric Configuration Example of Image Processing Device according to Third Embodiment

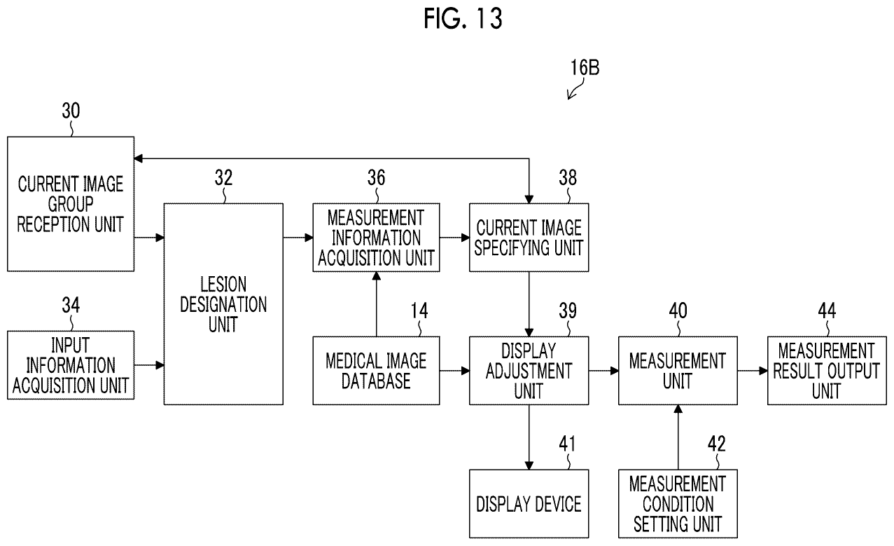

FIG. 13 is a functional block diagram showing an electric configuration of an image processing device according to the third embodiment. An image processing device 16B illustrated in FIG. 13 is different from the image processing device 16 illustrated in FIG. 4 in that a display adjustment unit 39 is added.

The display adjustment unit 39 acquires the display condition of the past image PI11 illustrated in FIG. 12 from the medical image database 14. The display adjustment unit 39 adjusts the display condition of the current image CI13 specified by the current image specifying unit 38 and displays the current image CI13A in which the display condition of the current image CI13 is adjusted on the display device 41. As the display device 41 illustrated in

FIG. 13, the display device 20B of the user terminal 20 illustrated in FIG. 1 can be applied.

### Hardware Configuration Example of Image Processing Device according to Third Embodiment

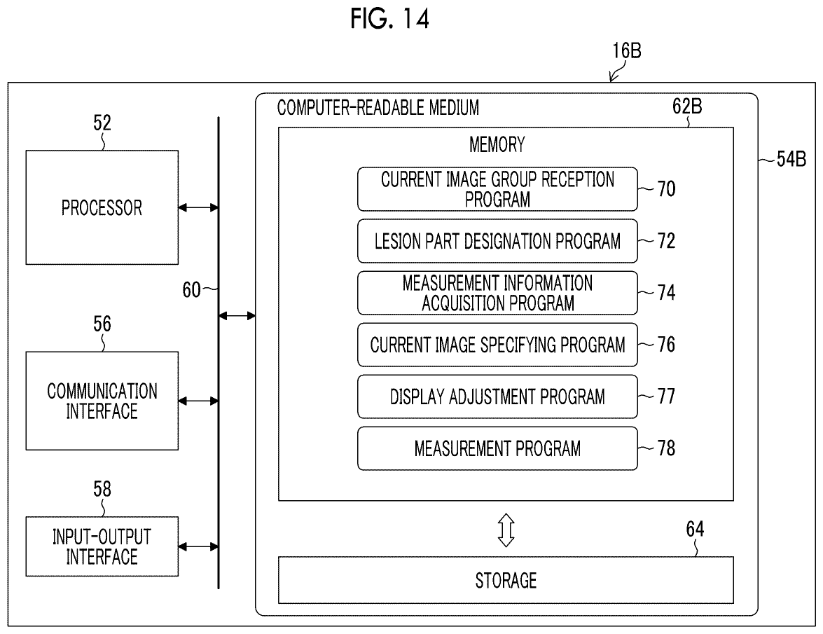

FIG. 14 is a block diagram schematically illustrating an example of a hardware configuration of an image processing system according to the third embodiment. The image processing device 16B illustrated in FIG. 14 comprises a computer-readable medium 54B instead of the computer-readable medium 54 of the image processing device 16 illustrated in FIG. 5. The computer-readable medium 54B comprises a memory 62B instead of the memory 62 illustrated in FIG. 5.

Various programs, such as a current image group reception program 70 stored in the memory 62 illustrated in FIG. 5, are stored in the memory 62B, and a display adjustment program 77 is added to the memory 62B. The display adjustment program 77 is applied to the display adjustment unit 39 illustrated in FIG. 13, and implements a display adjustment function.

### Actions and Effects of Third Embodiment

The image processing method and the image processing device according to the third embodiment can obtain the following actions and effects.

[1]

The display condition of the current image CI specified as the measurement target of the lesion part RI is adjusted in accordance with the display condition of the past image PI. Accordingly, the current image CI of the measurement target of the lesion part RI and the past image PI in which the lesion part is measured are displayed to have the same appearance, and in a case where the lesion part RI is measured, the request to use the same current image CI as the past image PI can be satisfied.

[2]

In a case where the past measurement information is acquired from the medical image database 14, the display condition of the past image PI corresponding to the past measurement information is acquired. Accordingly, it is possible to use the display condition of the past image PI with high reliability.

The technical scope of the present invention is not limited to the scope described in the above-mentioned embodiment. The configurations and the like in each embodiment can be appropriately combined between the respective embodiments without departing from the gist of the present invention.

### Explanation of References

10: medical image processing system
12: medical image examination device
14: medical image database
16: image processing device
16A: image processing device
16B: image processing device
20: user terminal
20A: input device
20B: display device
22: in-hospital network
24: Internet
26: cloud server
30: current image group reception unit
32: lesion designation unit
34: input information acquisition unit
36: measurement information acquisition unit
38: current image specifying unit
39: display adjustment unit
40: measurement unit
41: display device
42: measurement condition setting unit
44: measurement result output unit
52: processor
54: computer-readable medium
54A: computer-readable medium
54B: computer-readable medium
56: communication interface
58: input-output interface
60: bus
62: memory
62A: memory
62B: memory
64: storage
70: current image group reception program
72: lesion part designation program
74: measurement information acquisition program
76: current image specifying program
77: display adjustment program
78: measurement program
80: estimation error derivation unit
82: estimation error database
84: estimation error output unit
86: alert output unit
90: estimation error derivation program
92: alert output program
CI: current image
CI1: current image
CI2: current image
CI3: current image
CI11: current image
CI12: current image
CI13: current image
CI13A: current image
CI14: current image
CI21: current image
CI22: current image
CI24: current image
CIG: current image group
PI: past image
PI11: past image
PR: measurement result
RI: lesion part
S10 to S22: each step of image processing method

What is claimed is:

1. An image processing device comprising:
one or more processors; and
one or more memories that store a command to be executed by the one or more processors,
wherein the one or more processors are configured to:
receive a plurality of first images that are generated by imaging a subject and to which each of a plurality of first image conditions different from each other is applied;
designate a first region of interest in the plurality of first images;
acquire, for a plurality of second images that are generated by imaging the subject before capturing the first image, measurement information on the second image having a second region of interest corresponding to the first region of interest;
specify the first image included in a range of the measurement information from the plurality of first images; and
measure the first region of interest in the specified first image.

2. The image processing device according to claim 1, wherein the one or more processors are configured to specify the first image having the first image condition in which a similarity indicator representing a similarity to a second image condition of the second image is a maximum, as the first image included in the range of the measurement information.

3. The image processing device according to claim 2, wherein the one or more processors are configured to derive the similarity indicator using at least any one of a reconstruction condition, a slice thickness representing a thickness of a tomographic layer in a tomographic image, a spatial resolution, or an imaging protocol.

4. The image processing device according to claim 1, wherein the one or more processors are configured to apply at least any one of the second image in which measurement of the second region of interest is performed or a measurement result of the second region of interest, as the measurement information.

5. The image processing device according to claim 1, wherein the one or more processors are configured to estimate an error in a measurement result of the first region of interest with respect to a measurement result of the second region of interest, based on the first image condition and a second image condition.

6. The image processing device according to claim 5, wherein the one or more processors are configured to output an alert in a case where the estimated error exceeds a prescribed range.

7. The image processing device according to claim 5, wherein the one or more processors are configured to display a range of the error prescribed according to a tendency of the estimated error on a display device.

8. The image processing device according to claim 1, wherein the one or more processors are configured to set a first display condition applied to measurement of the first region of interest in the first image according to a second display condition applied to measurement of the second region of interest in the second image.

9. The image processing device according to claim 8, wherein the one or more processors are configured to set at least any one of brightness, a window level, or a window width as the first display condition.

10. The image processing device according to claim 1, wherein the one or more processors are configured to display at least any one of the first region of interest or a measurement result of the first region of interest on a display device in a case where an input for selecting the first region of interest in the first image is acquired.

11. The image processing device according to claim 1, wherein the one or more processors are configured to display at least one of the first region of interest or a measurement result of the first region of interest on a display device and to display the measurement information on the display device in a case where an input for selecting the first region of interest in the first image is acquired.

12. The image processing device according to claim 11, wherein the one or more processors are configured to apply different display aspects to at least any one of the first region of interest or the measurement result of the first region of interest, and the measurement information.

13. The image processing device according to claim 11, wherein the one or more processors are configured to display at least any one of the second image or a measurement result of the second region of interest as the measurement information on the display device in a case where an input for selecting the first region of interest in the first image is acquired.

14. The image processing device according to claim 1, wherein the one or more processors are configured to:
receive, as the plurality of first images, a plurality of first medical images generated by imaging a subject;
designate, as the first region of interest, an anatomical structure of the subject in the plurality of first medical images; and
acquire, as the measurement information, measurement information obtained by measuring the anatomical structure of the subject which is the second region of interest corresponding to the first region of interest for a plurality of second medical images generated by imaging the subject.

15. The image processing device according to claim 14, wherein the one or more processors are configured to display a measurement result of the anatomical structure on a display device as a measurement result of the first region of interest in the first image.

16. An operation method of an image processing device including one or more processors and one or more memories that store a program to be executed by the one or more processors, the method comprising:
via the one or more processors,
receiving a plurality of first images that are generated by imaging a subject and to which each of a plurality of first image conditions different from each other is applied;
designating a first region of interest in the plurality of first images;
acquiring, for a plurality of second images that are generated by imaging the subject before capturing the first image, measurement information on the second image having a second region of interest corresponding to the first region of interest;
specifying the first image included in a range of the measurement information from the plurality of first images; and
measuring the first region of interest in the specified first image.

17. A non-transitory, computer-readable tangible recording medium which records thereon a program for causing, when read by a computer, the computer to implement:
a function of receiving a plurality of first images that are generated by imaging a subject and to which each of a plurality of first image conditions different from each other is applied;
a function of designating a first region of interest in the plurality of first images;
a function of acquiring, for a plurality of second images that are generated by imaging the subject before capturing the first image, measurement information on the second image having a second region of interest corresponding to the first region of interest;
a function of specifying the first image included in a range of the measurement information from the plurality of first images; and
a function of measuring the first region of interest in the specified first image.
